SEQUENCE LISTING (2 sequences)

SEQ ID NO: 1 - DNA, 41 bp, Artificial Sequence
  rs3730089
  misc_feature (21)..(21): n is g or a
aacggtatgaataacaatatntccttacaagatgctgaatg

SEQ ID NO: 2 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

(19) United States
(12) Patent Application Publication
(10) Pub. No.: US 2019/0100806 A1
(43) Pub. Date: Apr. 4, 2019

(54) MARKER FOR PREDICTING TREATMENT RESPONSE TO ANTI-CANCER AGENT IN SOLID CANCER PATIENTS

(71) Applicants: DAEWOONG PHARMACEUTICAL CO., LTD., Gyeonggi-do (KR); INDUSTRY-ACADEMIC COOPERATION FOUNDATION, YONSEI UNIVERSITY, Seoul (KR)

(72) Inventors: Jaeyoung KIM, Seoul (KR); Jingmin CHE, Seoul (KR); Hyun Cheol CHUNG, Seoul (KR); Sun Young RHA, Seoul (KR); Woo Sun KWON, Gyeonggi-do (KR); Tae Soo KIM, Gyeonggi-do (KR)

(21) Appl. No.: 16/087,561

(22) PCT Filed: Mar. 23, 2016
(86) PCT No.: PCT/KR2016/002896

§ 371 Date: Sep. 21, 2018

(30) Foreign Application Priority Data
Mar. 22, 2016 (KR) 10-2016-0033813

(51) Int. Cl. C12Q 1/6886; G16H 50/20; G16H 50/30; C12Q 1/6827

(52) U.S. Cl. CPC C12Q 1/6886; C12Q 1/6827; G16H 50/30; G16H 50/20

(57) ABSTRACT

The present invention relates to a marker for predicting a responsiveness of a solid cancer patient to an anticancer agent. The marker according to the present invention can be advantageously used to select a subgroup, who effectively responds to an anticancer therapy with a specific anticancer agent, from among solid cancer patients, or to determine a therapy method for treatment of solid cancer patients.

### TECHNICAL FIELD

The present invention relates to a marker for predicting the treatment responsiveness of a solid cancer patient to an anticancer agent, and more particularly to a method for providing information on determining whether treatment with a PI3Kβ inhibitor is to be performed, by detecting an SNP in the PIK3R1 gene.

### BACKGROUND ART

Stomach cancer has a high incidence, especially in Asia, and is the leading cause of cancer-related deaths (Ferlay J, Shin H R, Bray F, Forman D, Mathers C, Parkin D M. Estimates of worldwide burden of cancer in 2008: GLOBOCAN 2008. Int J Cancer 2010; 127: 2893917). In Korea, it is estimated that 16.2% of cancer patients (20.3% of male cancer patients and 11.2% of female cancer patients) are Stomach cancer patients. The annual-standardized incidence of Stomach cancer is 61.2/100,000 for men and 23.9/100,000 for women (Jung, K W, Park S, Kong H J, Won Y J, Boo Y K, Shin H R, et al. Cancer Statistics in Korea: Incidence, Mortality and Survival in 2006-2007. J Korean Med Sci 2010; 25: 1113-21).

A signaling pathway with phosphatidylinositol-4,5-bisphosphate 3-kinase (PI3K) is one of signaling pathways with the most frequently occurring mutations in stomach cancer.

PI3K is an enzyme that converts phosphatidylinositol 4,5-bisphosphate to phosphatidylinositol 3,4,5-trisphosphate (PI(3,4,5)P3) which is an active signaling intermediate. PI(3,4,5)P3 activates pyruvate dehydrogenase kinase isozyme 1 (PDK1), and then activates Akt. PI3K consists of two subunits (p110 and p85) which are each divided into a plurality of subtypes. Focusing on one subunit, p110, it has two subtypes (PIK3CA and PIK3CB), which show a overlapping function. PTEN (phosphatase and tensin homolog deleted on chromosome 10) is a negative regulator of PI3K, which dephosphorylates PI (3,4,5) P3 and inhibits the PI3K signaling pathway. Activation of the PI3K signaling pathway is known to be caused by up-regulation of upstream receptor tyrosine kinase (RTK) signaling, such as a variant in PI3KCA (phosphoinositide-3-kinase, catalytic, alpha polypeptide) or PTEN deficiency. RTK activation of PI3K is known to transform cells and cause dependency on PIK3CA, and PTEN deficiency is also known to increase downstream Akt activity and PI3K activity, which act mainly through PI3KCB.

In PI3K/AKT signaling pathway PI3K activates PDK1 and Akt and transforms cells.

Meanwhile, it is known that inhibitors acting specifically on PI3K beta-isoform exhibit effects while having appropriate cytotoxic effects on cancer patients in whom PTEN protein is not expressed. In recent years, it has been reported that when the activity of PI3K beta-isoform is inhibited in an animal model in which PTEN is not expressed, cancer development can be effectively inhibited (Jia S et al., Nature. Vol. 454, pp 776-9, 2008; Wee S et al., PNAS. Vl. 105, pp 13057-62. 2008; Torbett N E et al., Biochem J. Vol. 415. pp 97-110. 2008; Jing Ni et al., Cancer Discovery. Vol. 5. pp 425-33. 2012).

When GSK2636771, an inhibitor that acts specifically on PI3K beta-isoform, is subjected to structure-activity relationship optimization based on a TGX-221 compound, a compound that selectively and strongly inhibits phosphatidylinositol 4,5-bisphosphate 3-kinase catalytic subunit beta isoform (PI3Kβ) can be discovered very rapidly. Substitution of this compound with benzimidazole led to potent PI3Kβ inhibition, and it was found to be a lead compound in this compound group (Rivero, R. A. et al. 103rd Annu Meet Am Assoc Cancer Res (AACR) (March 31-April 4, Chicago) 2012, Abst 2913).

In previous studies, PI3Kβ inhibition showed the effect of inhibiting tumor formation in phosphatidylinositol-3,4,5-trisphosphate 3-phosphatase and dual-specificity protein phosphatase PTEN-deficient tumors. It is known that GSK-2636771 inhibits the phosphorylation of RAC serine/threonine-protein kinase (Akt) in a dose-dependent manner in mouse models xenografted with human PTEN-deficient tumor cells (Hardwicke, M. A. et al. 243rd ACS Natl Meet (March 25-29, San Diego) 2012, Abst MEDI 21).

Furthermore, GSK-2636771 was reported to have median effective concentrations (EC50) of 36 nM and 72 nM against PC-3 (human prostate cancer) and HCC70 (human breast ductal carcinoma) cells, respectively, which are PTEN-deficient cells. It was reported that when mice were treated with 100 mg/kg of GSK-2636771, the PI3Kβ inhibitor did not increase glucose and insulin levels. In addition, a single dose of GSK-2636771 in a mouse model xenografted with PC-3 cells is known to reduce Akt phosphorylation (Ser473) (Wooster, R. 103rd Annu Meet Am Assoc Cancer Res (AACR) (March 31-April 4, Chicago) 2012, Abst), and GSK-2636771 is currently undergoing clinical trials.

However, a gene biomarker capable of predicting responsiveness to an inhibitor that acts specifically on PI3K beta-isoform has not been known yet.

Accordingly, the present inventors have made extensive efforts to develop a method capable of predicting responsiveness to an inhibitor that acts specifically on PI3K beta-isoform, and as a result, have found that when there is SNP (r53730089) in the PIK3R1 gene, an inhibitor that acts specifically on PI3K beta-isoform exhibits an excellent effect, thereby completing the present invention.

### DISCLOSURE OF INVENTION

### Technical Problem

It is an object of the present invention to provide a method for providing information for predicting the treatment responsiveness of a solid cancer patient to an anticancer agent.

Another object of the present invention is to provide a primer and/or probe composition for predicting the responsiveness of a solid cancer patient to an anticancer agent, and a kit for predicting the responsiveness of a solid cancer patient to an anticancer agent, the kit comprising the same.

Still another object of the present invention is to provide a method for screening a patient-specific therapeutic agent for treatment of solid cancer.

### Technical Solution

To achieve the above object, the present invention provides a method for providing information for predicting a responsiveness of a solid cancer patient to an anticancer agent, the method comprising detecting in a sample the presence or absence of an SNP (NCBI refSNP ID: r53730089) at nucleotide position 21 in the nucleotide sequence of SEQ ID NO: 1, which is a portion of a PIK3R1.

The present invention also provides a primer composition for predicting a responsiveness of a solid cancer patient to an anticancer agent, the primer composition comprising a primer or detecting a polynucleotide comprising 10 or more consecutive nucleotides including the 21^{st} nucleotide in the nucleotide sequence of SEQ ID NO: 1 (NCBI refSNP ID: r53730089), which is a portion of a PIK3R1 gene, or a complementary polynucleotide thereof.

The present invention also provides a probe composition for predicting a responsiveness of a solid cancer patient to an anticancer agent, the probe composition comprising a probe for hybridizing specifically to a polynucleotide comprising or more consecutive nucleotides including the 21^{st} nucleotide in the nucleotide sequence of SEQ ID NO: 1 (NCBI refSNP ID: r53730089), which is a portion of a PIK3R1 gene, or a complementary polynucleotide thereof.

The present invention also provides a method for screening a patient-specific therapeutic agent for treatment of solid cancer, the method comprising the step of: (a) detecting in a sample the presence or absence of an SNP (NCBI refSNP ID: r53730089) located at nucleotide position 21 in the nucleotide sequence of SEQ ID NO: 1, which is a portion of a PIK3R1 gene; and (b) when the SNP is present, selecting a phosphoinositide 3-kinase β (PI3Kβ) inhibitor as the patient-specific therapeutic agent.

### BRIEF DESCRIPTION OF THE DRAWINGS

FIG. 1 is a schematic view showing the overall flow of an experiment on predicting a responsiveness of an SNP, identified in the present invention, to an anticancer agent.

FIG. 2 summarizes the results of whole exome sequencing performed to detect variants in PI3K-related genes in 51 stomach cancer cell lines used in the present invention.

FIG. 3 depicts graphs summarizing a correlation between responsiveness to PI3Kβ inhibitors and gene variants in PI3K-related genes in 51 stomach cancer cell lines used in the present invention.

FIG. 4(A) shows the results of statistically analyzing the correlation between responsiveness to PI3Kβ inhibitors and PI3K-related genes' variants, and FIG. 4(B) shows the results of statistically analyzing the responsiveness of a PIK3R1 M326I variant to a PI3Kβ inhibitor.

FIG. 5 is a volcano graph showing the results of analysis performed to analyze responsiveness to a PI3Kβ inhibitor in the presence or absence of a PI3K-related gene variant and to analyze statistical significance. In addition, it shows the proportion of cell lines that respond to a PI3Kβ inhibitor in PIK3R1 wild-type cell lines or M326I variant cell lines.

FIG. 6 shows the results of analyzing the correlation between the heterozygosity of a PIK3R1 M326I variant and responsiveness to a PI3Kβ inhibitor.

FIG. 7 shows the results of three-dimensional conformational analysis performed to determine how a PIK3R1 M326I variant changes the conformation of PIK3R1 and thereby increases responsiveness to a PI3Kβ inhibitor.

FIG. 7A shows the results of analyzing the three-dimensional conformations of wild-type PIK3R1 and variant PIK3R1 and calculating each binding energy.

FIG. 7B shows three-dimensional models obtained by analyzing how a portion of wild-type PIK3R1 or variant PIK3R1 through which binds to GSK2636771 conformationally changes.

FIG. 8 shows that the SNP of the present invention can be used to predict responsiveness to a PI3Kβ inhibitor with high accuracy.

FIG. 9 is a conceptual view showing a method for screening a patient-specific therapeutic agent for treatment of solid cancer according to the present invention.

FIG. 10 shows that the use of the SNP of the present invention makes it possible to measure responsiveness to a PI3Kβ inhibitor not only in Stomach cancer, but also in various solid cancers.

### BEST MODE FOR CARRYING OUT THE INVENTION

Unless defined otherwise, all the technical and scientific terms used herein have the same meaning as those generally understood by one of ordinary skill in the art to which the invention pertains. Generally, the nomenclature used herein and the experiment methods, which will be described below, are those well known and commonly employed in the art.

In the present invention, efforts have been made to develop a method capable of predicting responsiveness of solid cancer patients to anticancer agents and to confirm the accuracy of the prediction.

In the present invention, information on PI3K-related genes' variants in a variety of stomach cancer cell lines was identified by whole exome sequencing, and responsiveness of each cell line to a PI3Kβ inhibitor was determined by a cell viability assay, and then a PI3K-related gene variant associated with responsiveness to the PI3Kβ inhibitor was selected by statistical analysis.

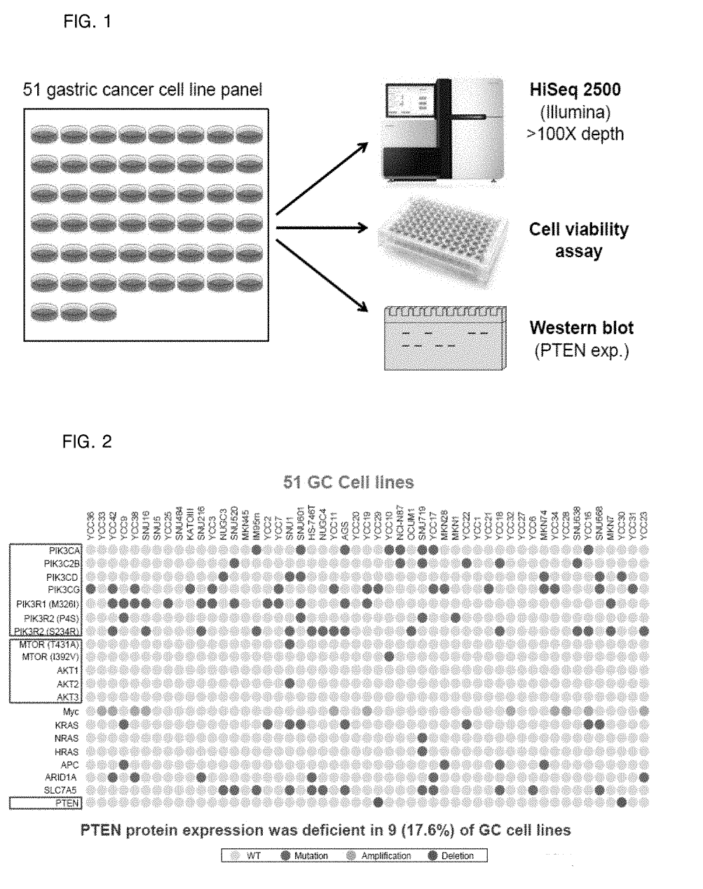

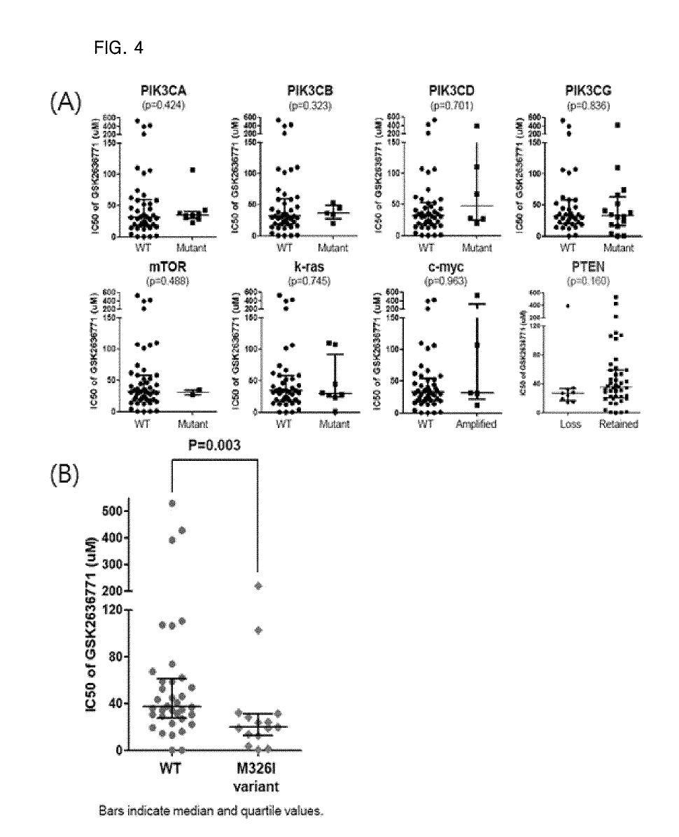

Specifically, in one example of the present invention, information on PI3K-related genes' variants in 51 stomach cancer cell lines was identified by whole exome sequencing, and then a cell viability assay was performed using a PI3Kβ inhibitor, and the expression level of PTEN protein associated with responsiveness to the PI3Kβ inhibitor was also analyzed (FIGS. 1 to 3). As a result, it was found that a PIK3R1 M326I variant (NCBI refSNP ID:rs3730089) can predict responsiveness to the PI3Kβ inhibitor (FIGS. 4 to 6).

Therefore, in one aspect, the present invention is directed to a method for providing information for predicting responsiveness of a solid cancer patient to an anticancer agent, the method comprising detecting in a sample the presence or absence of an SNP (NCBI refSNP ID: r53730089) at nucleotide position 21 in the nucleotide sequence of SEQ ID NO: 1, which is a portion of a PIK3R1 gene.

The sequence is shown in the following SEQ ID NO: 1. NCBI refSNP ID informs the sequence and position of SNP. A person skilled in the art can easily identify the position and the sequence of the SNP by using NCBI refSNP ID of the SEQ ID NO: 1. It will be obvious to a person having ordinary skill in the art that the specific sequence corresponding to the refSNP ID of SNP, registered in NCIB, may be modified slightly depending on the results of the successive studies on the gene, and such sequence modification also falls within the scope of the present invention:

SEQ ID NO: 1: rs3730089
AACGGTATGA ATAACAATAT[G/A]TCCTTACAAG ATGCTGAATG.

The identification of the genotype of SNP of the present invention can be performed by any methods known in the art, such as a general sequencing analysis, sequencing analysis using an automatic nucleotide sequence analyzer, pyrosequencing, hybridization by microarray, a PCR-based restriction fragment length polymorphism (PCR-RELP) method, a PCR-single strand conformation polymorphism (PCR-SSCP) method, a PCR-specific sequence oligonucleotide (PCR-SSO) method, allele-specific oligonucleotide (ASO) hybridization method which is a combination of PCR-SSO method and dot hybridization method, a TaqMan-PCR method, an MALDI-TOF/MS method, a rolling circle amplification (RCA) method, a high resolution melting (HRM) method, a primer extension assay, a Southern blot hybridization method, and a dot hybridization method. Furthermore, the results of the SNP polymorphism can be statistically processed using a statistical analysis method commonly used in the art, and can be analyzed by using continuous variables, categorical variables, and variables such as odds ratios and 95% confidence intervals, which are obtained through, for example, Student's t-test, Chi-square test, linear regression line analysis, multiple logistic regression analysis and the like.

As used herein, the term “predicting” is related to whether a patient will survive or have a possibility to survive after chemotherapeutic treatment and the like, and/or surgical removal of primary tumors by responding preferentially or non-preferentially to therapy, and/or whether a patient will survive or have a possibility to survive without cancer recurrence after the chemotherapeutic treatment and/or the surgery.

The prediction method of the present invention may be clinically used to determine treatment by selecting the most suitable therapeutic method for a solid cancer patient. In addition, the prediction method of the present invention can predict whether a patient will preferentially response to therapeutic treatments, including a specific therapeutic agent or a combination therapy, surgical intervention, chemotherapy, and the like, or whether a patient can survive for a long period of time after the therapeutic treatment.

In another aspect, the present invention is directed to a primer composition for predicting responsiveness of a solid cancer patient to an anticancer agent, the primer composition comprising a primer for detecting a polynucleotide comprising or more consecutive nucleotides including the 21^{st} nucleotide of SEQ ID NO: 1 (NCBI refSNP ID: r53730089), which is a portion of a PIK3R1 gene, or a complementary polynucleotide thereof.

In the present invention, appropriate length of the primer may vary depending on the use, but can generally be composed of 15 to 30 nucleotides. A primer sequence is not necessarily completely complementary with a template but must be complementary enough to hybridize with the template. The primer can hybridize to DNA sequences containing a polymorphic site(s) to amplify DNA fragments containing a polymorphic site(s). The primer of the present invention can be used in a diagnostic kit or a prediction method for predicting responsiveness of a solid cancer patient to an anticancer agent by detecting an allele.

In still another aspect, the present invention is directed to a probe composition for predicting responsiveness of a solid cancer patient to an anticancer agent, the probe composition comprising a probe for hybridizing specifically to a polynucleotide comprising 10 or more consecutive nucleotides including the 21^{st} nucleotide in the nucleotide sequence of SEQ ID NO: l(NCBI refSNP ID: r53730089), which is a portion of a PIK3R1 gene, or a complementary polynucleotide thereof.

In the present invention, the probe may be allele-specific. This means that the probe hybridizes specifically to each allele. Namely, this means that the probe hybridizes specifically to each allele so that it can specifically detect a nucleotide at a polymorphic site present in a polymorphic sequence. Here, the hybridization is usually performed under stringent conditions, for example, at a salt concentration of 1M or less and a temperature of 25° C. or higher. For example, the conditions of 5×SSPE (750 mM NaCl, 50 mM Na Phosphate, 5 mM EDTA, pH 7.4) and 25 to 30° C. may be suitable for allele-specific probe hybridization.

In the present invention, the probe means a hybridization probe and an oligonucleotide capable of sequence-specifically binding to a complementary strand of nucleic acids. The allele-specific probe of the present invention can hybridize to a fragment of target DNA from one individual, but may not hybridize to the corresponding fragment from another individual due to the presence of a polymorphic site in the respective nucleic acid fragments from the two individuals of the same species. In this case, hybridization conditions should be sufficiently stringent so that there is a significant difference in hybridization intensity between alleles, and thus the probe hybridizes to only one of the alleles. This probe of the present invention is preferably designed such that the central position aligns with the polymorphic site of the polymorphic sequence. This probe design can induce good discrimination in hybridization between different allelic forms. The probe of the present invention can be used in a diagnostic kit or a prediction method for predicting responsiveness of a solid cancer patient to an anticancer agent by detecting an allele.

In still another aspect, the present invention is also directed to a composition for predicting responsiveness of a solid cancer patient to an anticancer agent, the composition comprising an antibody or an aptamer that specifically binds to a polypeptide encoded by a polynucleotide including the 21^{st} nucleotide in the nucleotide sequence of SEQ ID NO: 1 (NCBI refSNP ID: r53730089), which is a portion of a PIK3R1 gene.

In still another aspect, the present invention is directed to a kit for predicting responsiveness of a solid cancer patient to an anticancer agent, the kit comprising any one of the above-described compositions of the present invention.

In the present invention, the kit may comprise, in addition to the polynucleotide, the antibody or the aptamer of the present invention, one or more constituent compositions, solutions or devices suitable for the analysis method. In one embodiment, the kit of the present invention may be a kit which comprises essential elements necessary to perform a PCR. The kit may further include a test tube or other appropriate container, a reaction buffer (various pHs and magnesium concentrations), deoxynucleotides (dNTPs), enzymes such as Taq-polymerase and reverse transcriptase, a DNAse inhibitor, a RNAse inhibitor, DEPC-water, or sterilized water, etc. In another embodiment, the kit of the present invention may be a kit for predicting prognosis of solid cancer, which comprises essential elements required for performing a DNA chip assay. The DNA chip kit may comprise a substrate having immobilized thereon a polynucleotide, primer or probe specific for the SNP. In addition, the substrate may comprise a nucleic acid corresponding to a quantitative control gene or its fragment.

In the present invention, the anticancer agent can be used without any limitation as long as it is a drug that can inhibits solid cancers. Preferably, the anticancer agent may be a phosphoinositide 3-kinase β (PI3Kβ) inhibitor, and may more preferably by selected from the group consisting of GSK2636771, SAR260301, TGX-221, AZD5482, and KIN-193.

In the present invention, the solid cancer may be selected from the group consisting of stomach cancer, liver cancer, glioblastoma, ovarian cancer, colon cancer, head and neck cancer, bladder cancer, renal cell cancer, breast cancer, metastatic cancer, prostate cancer, pancreatic cancer, melanoma, and lung cancer, but is not limited thereto.

In yet another aspect, the present invention is directed to a method for screening a patient-specific therapeutic agent for treatment of solid cancer, the method comprising the steps of: (a) detecting in a sample the presence or absence of an SNP (NCBI refSNP ID: r53730089) located at nucleotide position 21 in the nucleotide of SEQ ID NO: 1, which is a portion of a PIK3R1 gene; and (b) when the SNP is present, selecting a phosphoinositide 3-kinase β (PI3Kβ) inhibitor as the patient-specific therapeutic agent.

In the present invention, the method may further comprise, after step (a), a step of measuring the expression level of PTEN protein.

The expression level of the PTEN protein of the present invention can identify the amount of protein using an antibody that specifically binds to the protein of the gene. Analysis methods for measuring the amount of the protein using an antibody include, but are not limited to, Western blotting, ELISA (Enzyme Linked Immunosorbent Assay), radioimmunoassay (RIA), radioimmunodiffusion, rocket immunoelectrophoresis, immunohistostaining, immunoprecipitation assay, complement fixation assay, FACS, protein chip assay, etc.

In the present invention, the expression level of the PTEN protein can be analyzed by measuring the amount of mRNA, and analysis methods for measuring the expression level of mRNA include, but are not limited to, DNA chip assay, reverse transcription-PCR (RT-PCR), competitive RT-PCR, real-time PCR, RNase protection assay (RPA), Northern blotting, etc.

In the present invention, the method may further comprise, before step (a), the steps of: (a) detecting in a sample the presence or absence of a variant of phosphatidylinositol 4,5-bisphosphate 3-kinase catalytic subunit alpha (PIK3CA); and (b) when the variant is present, selecting a phosphoinositide 3-kinase α (PI3Kα) inhibitor as the patient-specific therapeutic agent.

In the present invention, the variant of PIK3CA may be selected from the group consisting of, but not limited to, P140R, I381M, E453K, E542K, E545K, and H1047R in PIK3CA having the amino acid sequence of SEQ ID NO: 2. In the present invention, the presence or absence of the variant of PIK3CA may be detected by a method using antibodies specific for each variant, sequencing, PCR and the like.

In the present invention, the phosphoinositide 3-kinase alpha (PI3Kα) inhibitor may be selected from the group consisting of, but not limited to, HS-173, Alpelisib (BYL719), CH5132799, Gedatolisib (PF-05212384, PKI-587), PIK-75, A66, and YM201636.

In the present invention, the sample can be used without any limitation as long as it is a gene sample derived from a patient. The gene sample may be DNA or RNA. The gene sample derived from a patient means a gene sample isolated from a patient's blood, tissue sample, feces, urine, or sputum.

A method of isolating the genome DNA from a patient to obtain the gene sample of the present invention may be performed by methods known in the art. For example, the method of the present invention may be performed either by purifying DNA directly from tissue, blood or cells, or by specifically amplifying a specific region of DNA by an amplification method such as PCR and isolating the amplification product. As used herein, the term “DNA” is meant to include not only DNA, but also cDNA synthesized from mRNA. A step of obtaining a nucleic acid from a subject may be performed using, for example, PCR amplification, ligase chain reaction (LCR), transcription amplification, self-sustained sequence replication, or nucleic acid sequence-based amplification (NASBA).

### EXAMPLES

Hereinafter, the present invention will be described in further detail with reference to examples. It will be obvious to a person having ordinary skill in the art that these examples are for illustrative purposes only and are not to be construed to limit the scope of the present invention.

### Example 1: Whole Exome Sequencing Analysis of Stomach Cancer Cell Lines

Sequencing analysis is a method of analyzing the whole DNA of a living organism. High-throughput sequencing is a method of analyzing all DNA sequences that make up over 90% of the genome, including protein-encoding regions. High-throughput genomic sequences provide only sequence information consisting of four nucleotides (A, T, G, C) and information showing the quality of the nucleotides. From sequence information on nucleotide sequences, the locations and structures of genes can be identified. To identify the locations of genes, in high-throughput sequencing, reads are made from very long DNA molecules, and then the overlapping regions of short read sequences are connected, after which the locations of genes are identified by bioinformatic techniques. In the present invention, a resequencing method was used which detects the structures and variants of genes by comparing the high-throughput read sequences of a specific organism with an established human genome reference sequence. Whole exome sequencing is one of target sequencing methods for analyzing a portion of a specific genome, and is a method for sequencing protein-encoding exon regions. The human genome has about 180,000 exons, the total length of which is about 30 MB, which corresponds to about 1% of the human genome.

To analyze the WESs of 51 stomach cancer cell lines, gDNA was extracted, and the QC for the gDNA was identified using the Agilent 2200 TapeStation System. Sample purification was performed using the Agencourt AMPure XP kit. For a library to perform WES sequencing, the SureSelect Library Prep Kit (Agilent) was used. To capture the whole exome of the human genome, the SureSelect Automated Hybridization Kit (Agilent) was used. Sequencing was performed on HiSeq 2500 (Illumina), with a 150-bp paired end running. Analysis for each variant was performed using Varscan2.3.5.

To detect a PIK3R1 M326I variant, an association study was used to analyze the correlation between the PIK3R1 M326I genotype and the phenotype for the drug sensitivity of PI3Kβ inhibitors. This method is a method for determining genes that are present at high frequencies in a population that responds to a PI3Kβ inhibitor rather than to a group that does not respond to the PI3Kβ inhibitor.

As a result, as shown in Table 1 below and FIG. 2, gene variants occurred in 51 stomach cancer cell lines.

TABLE 1
List of PI3K gene-related variants that
occurred in stomach cancer cell lines

| | No. of altered | | | |
|---|---|---|---|---|
| | cell | Somatic | | |
| gene | lines (N, %) | mutation | Polymorphism | CNV |
| PIK3CA | 8 (15.7%) | P140R (1), | None | None |
| | | I391M (2), | | |
| | | E453K (1), | | |
| | | E542K (2), | | |
| | | E545K (3), | | |
| | | H1047R (1) | | |
| PIK3CB | 1 (2.0%) | None | None | Copy loss |
| | | | | in YCC-30 |
| PIK3C2B | 6 (11.8%) | R1366L (1), | None | None |
| | | T1360I (1), | | |
| | | T879N (1), | | |
| | | P717L (1), | | |
| | | P311L (1), | | |
| | | R250Q (1) | | |
| PIK3CD | 6 (11.8%) | T456A (5), | | None |
| | | T465M (1) | | |
| PIK3CG | 16 (31.4%) | R90W (2), | S442Y (13) | None |
| | | G436S (1), | | |
| | | A621S (2), | | |
| | | T857A (2) | | |
| PIK3R1 | 14 (27.5%) | None | M326I (14) | None |
| PIK3R2 | 19 (37.3%) | P4S (4) | S234R (15) | None |
| mTOR | 2 (3.9%) | T421A (1), | None | None |
| | | I392V (1) | | |
| AKT2 | 1 (2.0%) | T310M (1) | None | None |
| Myc | 11 (21.6%) | None | None | Amplified |
| | | | | in 11 |
| | | | | cell |
| | | | | lines |
| ARID1A | 6 (11.8%) | G444S (1), | None | None |
| | | R693X (1), | | |
| | | Q1458X | | |
| | | (1), P1771S | | |
| | | (1), D1912N | | |
| | | (1), K1907X | | |
| | | (1) | | |
| PTEN | 2 (3.9%) | None | None | Deleted |
| | | | | in 2 cell |
| | | | | lines |

### Example 2: Identification of PI3Kp Inhibitor-Associated Gene Variants

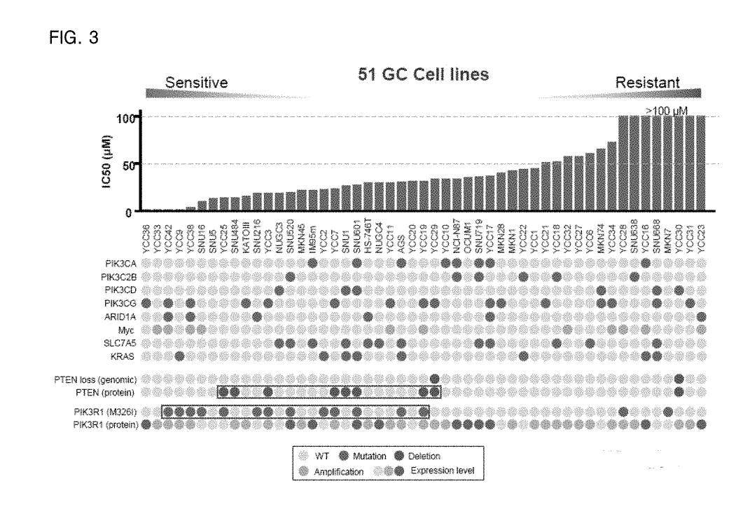

The responsiveness of stomach cancer cell lines to PI3Kβ inhibitors was examined. 51 stomach cancer cell lines were treated with various concentrations (0.001 to 100 μM) of a PI3Kβ inhibitor and incubated for 72 hours, and then the cell viability of the cell lines was measured by an MTT assay. From the measured cell viability, IC50 (inhibitory concentration 50) was calculated using CalcuSyn Version 2.0 (Biosoft) program. The calculated IC50 values were sorted in lower-value orders (sensitive) and compared with those of PI3K-related genes variants (FIG. 3). The mean of the IC50 values of the PI3Kβ inhibitor was compared between the two groups depending on the presence or absence of variants in the PI3K-related gene (FIG. 4). Comparison of the mean between the two groups was performed by the independent samples T test method (IBM SPSS Statistics 20). As a result, it was shown that when the PIK3R1 M326I variant was present in the PI3K-related gene, the IC50 value of the PI3Kβ inhibitor was lower compared to when the wild type was present(p=0.003).

### Example 3: Identification of Correlation Between PIK3R1 M326I Variant and PI3Kp Inhibitor

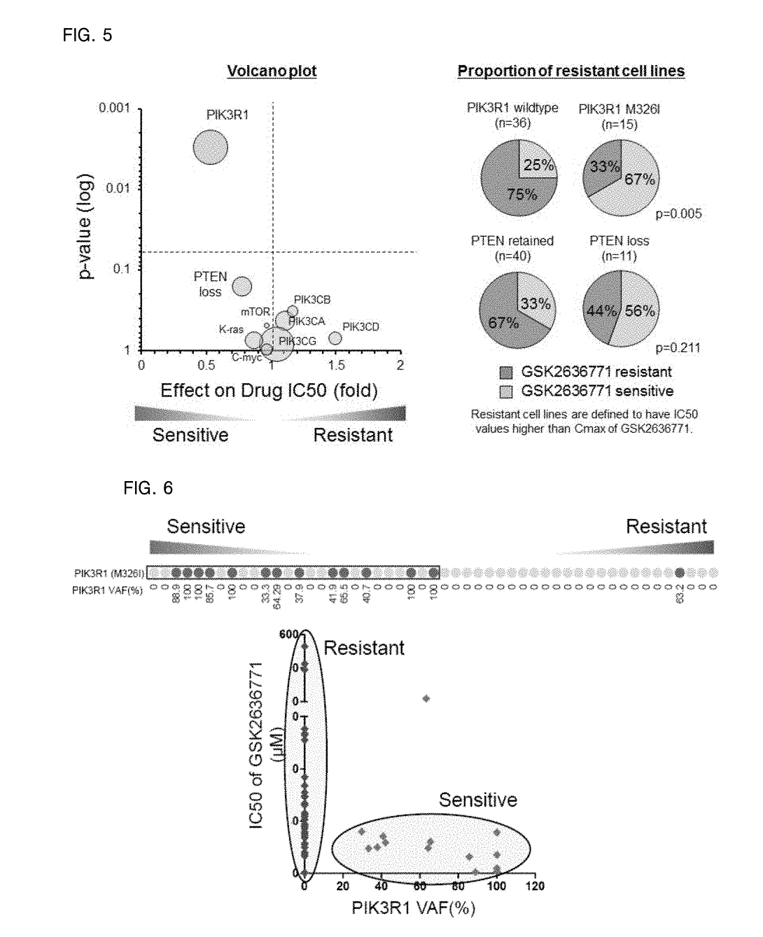

In order to effectively visualize the correlation between variants of PI3K-related genes and the IC50 of the PI3Kβ inhibitor, a volcano plot was prepared with reference to data published by the Sanger Institute (FIG. 5). It was shown that when the PIK3R1 M326I variant was present among PI3K-related genes, responsiveness to the PI3Kβ inhibitor was statistically significantly better.

In addition, responsiveness to the PI3Kβ inhibitor according to the variant allele frequency (VAF) of the PIK3R1 gene allele was analyzed, and as a result, the correlation between the two factors could not be seen (FIG. 6).

### Example 4: Analysis of Mechanism for Responsiveness of PIK3R1 M326I Variant to PI3Kp Inhibitor

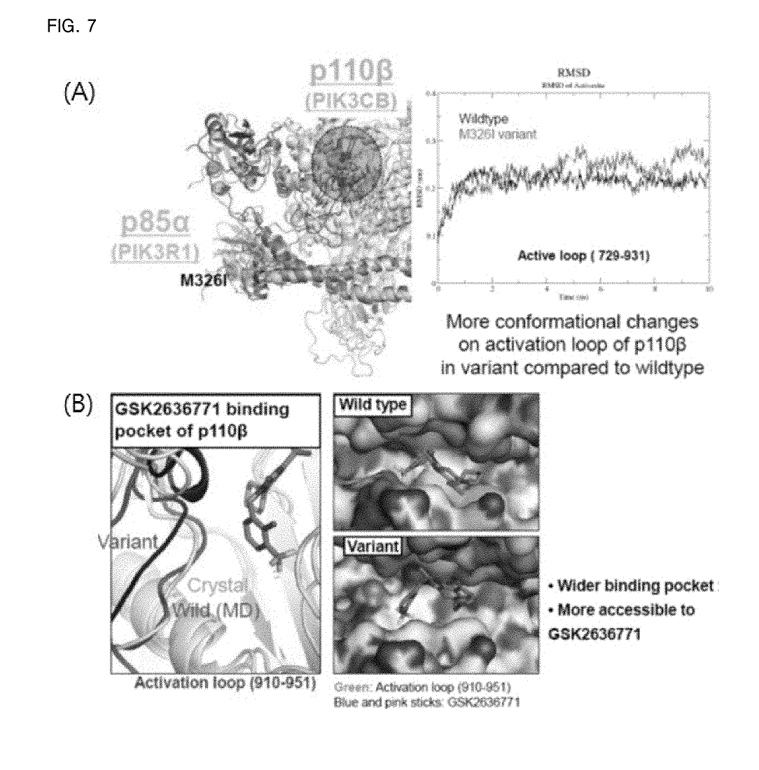

The effect of the PIK3R1 M326I variant on the PIK3R1 protein structure was analyzed in silico. The amino acid at position 326 of PIK3R1 is located near the nSH2 domain capable of regulating the activity of p110 protein. Therefore, it was confirmed through the results of the in silico analysis that when the M326I variant was present, the active conformation of the p110 protein resulting from the binding of PIK3R1(p85) to p110 is changed(FIG. 7). It is expected that the inhibitory function of p110 will be weaker in the PIK3R1 variant than in the wild-type. In addition, in the binding between the PIK3R1 M326I variant and PI3Kβ (p110β), the binding affinity of the PI3Kβ inhibitor for the ATP binding domain of PIK3a increased (FIG. 7 and Table 2). Due to this phenomenon, it was determined that when the PIK3R1 M326I variant was present, responsiveness to the PI3Kβ inhibitor would be better.

TABLE 2
Results of calculation of binding energy
between PI3K inhibitor and each of wild-type PI3K and variant
PI3K

| Binding free | | Variant |
|---|---|---|
| energies | Wild p110β/p85α/GSK | p110β/p85α/GSK |
| van der Waal | −172.298 ± 16.638 | −173.943 ± 8.257 |
| energy | | |
| Electrostatic | −97.561 ± 14.082 | 127.492 ± 14.275 |
| energy | | |
| Polar solvation | 194.650 ± 33.346 | 127.492 ± 14.275 |
| energy | | |
| SASA energy | −18.962 ± 0.731 | −18.962 ± 0.731 |
| Average binding | −94.172 ± 18.270 | −100.988 ± 13.009 |
| energy | (−22.50765 kcal/mol) | (−24.13 kcal/mol) |

### Example 5: Prediction of Responsiveness to PI3Kβ Inhibitor Depending on Biomarker

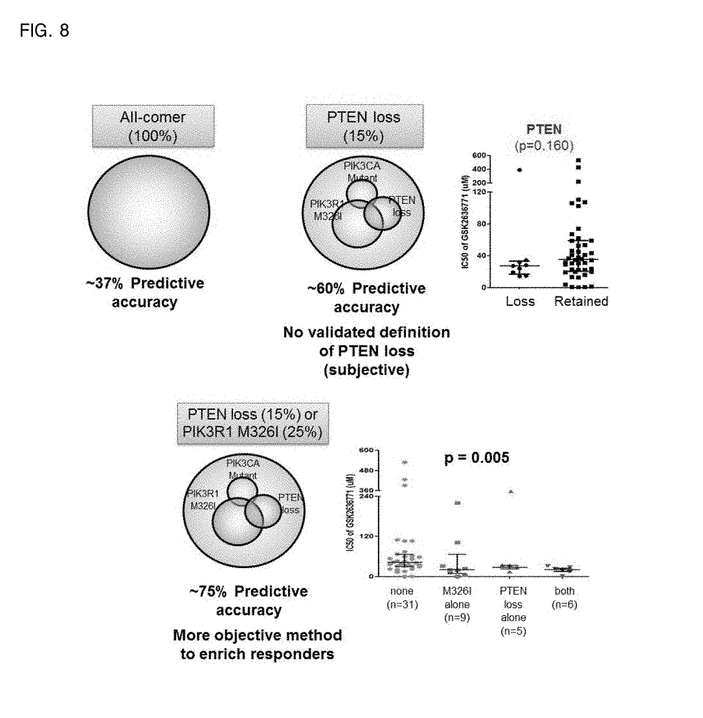

The responsiveness of an all-comer group to the PI3Kβ inhibitor was examined, and as a result, the responsiveness was predicted with a low accuracy of about 37%. The percentage showing loss or low expression of PTEN protein, a previously known predictive marker of responsiveness to the PI3Kβ inhibitor, was about 15% of the all-comer group, and the predictive accuracy of responsiveness of the PI3Kβ inhibitor to this group (PTEN loss) was about 60%, which was higher than that in the analysis for the all-comer group. However, when responsiveness to the PI3Kβ inhibitor was analyzed using a combination of the previously known PTEN loss with the PIK3R1 M326I gene variant identified in the present invention, the subject group increased up to 35%, and the predictive accuracy of responsiveness of this group to the PI3Kβ inhibitor was about 75%, which was higher than that in the analysis for the all-comer group or the PTEN loss group (FIG. 8).

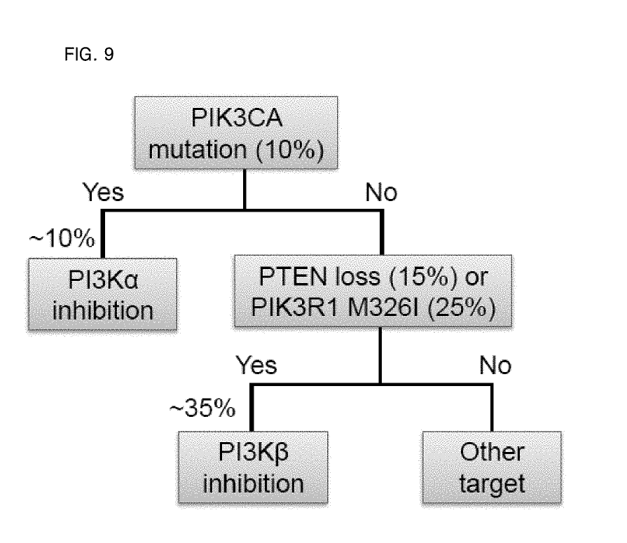

Thus, these study results suggest that, for a patient group having the PIK3CA variant, which is 10% of all cancer groups, treatment with the PI3Kα inhibitor should be performed, and for PTEN loss patients (about 15%) or PIK3R1 M326I variant patients (about 25%) among the remaining 90% of PIK3CA variant-negative patients, treatment with the PI3Kβ inhibitor should be performed (FIG. 9)

### Example 6: Identification of Correlation Between PIK3R1 M326I Variant and PI3Kp Inhibitor in Cancer Patients with Various Cancers

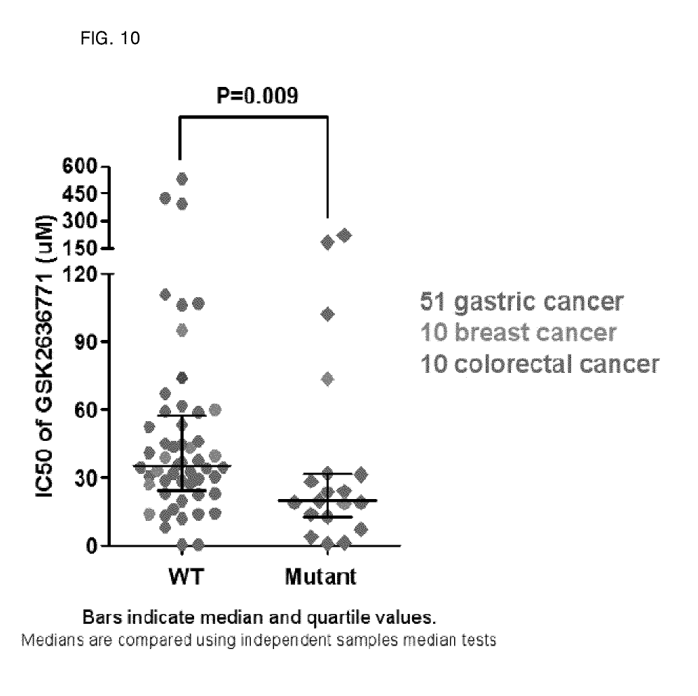

In addition to the 51 stomach cancer cell lines, the correlation between the PIK3R1 M326I variant and the PI3Kβ inhibitor was analyzed on 10 colorectal cancer cell lines and 10 breast cancer cell lines. As a result, it was shown that in the case of the PIK3R1 M326I variant was present, responsiveness of colorectal cancer and breast cancer to the PI3Kβ inhibitor was better than that in the case of the wild-type, in the same manner as the results of analysis on the stomach cancer cell lines (FIG. 10).

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### INDUSTRIAL APPLICABILITY

The method of detecting an SNP in the PIK3R1 gene according to the present invention can predict whether or not a specific anticancer agent will act on a solid cancer patient effectively, so that the method can be advantageously used to select a subgroup, who effectively responds to an anticancer therapy with a specific anticancer agent, from among solid cancer patients, or to determine a therapy method for treatment of solid cancer patients.

1. A method for providing information for predicting responsiveness of a solid cancer patient to an anticancer agent, the method comprising detecting in a sample the presence or absence of an SNP (NCBI refSNP ID: rs3730089) at nucleotide position 21 in the nucleotide sequence of SEQ ID NO: 1, which is a portion of a PIK3R1 gene.

2. The method of claim 1, wherein the anticancer agent is a phosphoinositide 3-kinase β (PI3Kβ) inhibitor.

3. The method of claim 2, wherein the phosphoinositide 3-kinase β (PI3Kβ) inhibitor is selected from the group consisting of GSK2636771, SAR260301, TGX-221, AZD5482, and KIN-193.

4. The method of claim 1, wherein the solid cancer is selected from the group consisting of stomach cancer, liver cancer, glioblastoma, ovarian cancer, colon cancer, head and neck cancer, bladder cancer, renal cell cancer, breast cancer, metastatic cancer, prostate cancer, pancreatic cancer, melanoma, and lung cancer.

5. A primer composition for predicting responsiveness of a solid cancer patient to an anticancer agent, the primer composition comprising a primer for detecting a polynucleotide comprising 10 or more consecutive nucleotides including the 21^{st} nucleotide in the nucleotide sequence of SEQ ID NO: 1, which is a portion of a PIK3R1 gene, or a complementary polynucleotide thereof.

6. A probe composition for predicting responsiveness of a solid cancer patient to an anticancer agent, the probe composition comprising a probe for hybridizing specifically to a polynucleotide comprising 10 or more consecutive nucleotides including the 21^{st} nucleotide in the nucleotide sequence of SEQ ID NO: 1, which is a portion of a PIK3R1 gene, or a complementary polynucleotide thereof.

7. A composition for predicting responsiveness of a solid cancer patient to an anticancer agent, the composition comprising an antibody or an aptamer that specifically binds to a polypeptide encoded by a polynucleotide comprising the SNP (NCBI refSNP ID: rs3730089) of claim 1.

8. The composition of any one of claims 5 to 7, wherein the anticancer agent is a phosphoinositide 3-kinase β (PI3Kβ) inhibitor.

9. The composition of claim 8, wherein the phosphoinositide 3-kinase β (PI3Kβ) inhibitor is selected from the group consisting of GSK2636771, TGX-221, AZD5482, and KIN-193.

10. A kit for predicting responsiveness of a solid cancer patient to an anticancer agent, the kit comprising the primer of claim 5, the probe of claim 6, or the antibody or aptamer of claim 7.

11. A method for screening a patient-specific therapeutic agent for treatment of solid cancer, the method comprising the step of:
(a) detecting in a sample the presence or absence of an SNP (NCBI refSNP ID: rs3730089) at nucleotide position 21 in the nucleotide sequence of SEQ ID NO: 1, which is a portion of a PIK3R1 gene; and
(b) when the SNP is present, selecting a phosphoinositide 3-kinase β (PI3Kβ) inhibitor as the patient-specific therapeutic agent.

12. The method of claim 11, wherein the phosphoinositide 3-kinase β (PI3Kβ) inhibitor is selected from the group consisting of GSK2636771, TGX-221, AZD5482, and KIN-193.

13. The method of claim 11, further comprising, after step (a), a step of measuring the protein level of PTEN.

14. The method of claim 11, further comprising, before step (a), the steps of:
(a) detecting in a sample the presence or absence of a variant of phosphatidylinositol 4,5-bisphosphate 3-kinase catalytic subunit alpha (PIK3CA) in a sample; and
(b) when the variant is present, selecting a phosphoinositide 3-kinase α (PI3Kα) inhibitor as the patient-specific therapeutic agent.

15. The method of claim 14, wherein the variant of PIK3CA is selected from the group consisting of P140R, I381M, E453K, E542K, E545K, and H1047R in PIK3CA.

16. The method of claim 14, wherein the phosphoinositide 3-kinase alpha (PI3Kα) inhibitor is selected from the group consisting of HS-173, Alpelisib (BYL719), CH5132799, Gedatolisib (PF-05212384, PKI-587), PIK-75, A66, and YM201636.

17. The method of claim 11, wherein the solid cancer is selected from the group consisting of stomach cancer, liver cancer, glioblastoma, ovarian cancer, colon cancer, head and neck cancer, bladder cancer, renal cell cancer, breast cancer, metastatic cancer, prostate cancer, pancreatic cancer, melanoma, and lung cancer.

18. The method of claim 1, wherein the sample is a gene sample derived from a patient.

19. The method of claim 11, wherein the sample is a gene sample derived from a patient.
